(19) United States
(12) Patent Application Publication
(10) Pub. No.: US 2011/0306789 A1
(43) Pub. Date: Dec. 15, 2011

(54) PROCESS FOR THE MANUFACTURE OF ADIPIC ACID

(75) Inventors: Philippe Carvin, Lyon (FR); Fabien Bellenger, Shanghai (CN); Serge Crottier-Combe, Diemoz (FR)

(73) Assignee: RHODIA OPERATIONS, Aubervilliers (FR)

(21) Appl. No.: 13/132,223

(22) PCT Filed: Nov. 27, 2009
(86) PCT No.: PCT/EP2009/065995

§ 371 Date: Jul. 28, 2011

(30) Foreign Application Priority Data
Dec. 1, 2008 (FR) 0806728

(51) Int. Cl. C07C 51/27

(52) U.S. Cl. 562/528; 562/540

(57) ABSTRACT

A process for the manufacture of adipic acid carried out in devices made of material resistant to the corrosion of the synthesis medium is described. A process for the manufacture of adipic acid carried out in devices, some of which are made of a material resistant to corrosion by nitric acid, is also described. The corrosion-resistant material can be a “duplex” austenoferritic stainless steel of X2 CrNiN 23-4 (1.4362) type, according to the European nomenclature.

The present invention relates to a process for the manufacture of adipic acid carried out in devices made of material resistant to the corrosion of the synthesis medium.

Adipic acid is an important chemical compound used as starting material in the manufacture of numerous products and in numerous applications. Thus, adipic acid is used as intermediate compound in the synthesis of macromolecular compounds, such as polyamides, more particularly PA 6/6, polyesters or polyurethanes, for example. This item is also used in the manufacture of plasticizers or as additives.

Several processes for the manufacture of adipic acid are known and made use of industrially. Currently, the main process made use of industrially consists in oxidizing cyclohexane with oxygen to obtain cyclohexanol and cyclohexanone intermediate compounds, generally a mixture of these two compounds. In a subsequent stage, the cyclohexanol/cyclohexanone intermediate compounds are oxidized by nitric acid in the presence of a catalyst to give adipic acid.

The adipic acid is recovered and purified by standard techniques, preferably by crystallization.

The reaction for the oxidation of the intermediate compounds by nitric acid is carried out in devices and reactors which have to be resistant to corrosion by nitric acid. Generally, these devices and reactors are constructed of special steels marketed for their resistance to corrosion by nitric acid.

However, under the temperature conditions and according to the nature of the compounds present in the oxidation medium of the process for the manufacture of adipic acid, a not insignificant phenomenon of corrosion of these special steels is observed.

This corrosion phenomenon exhibits disadvantages for the reliability of the equipment but in particular introduces metal impurities into the adipic acid produced. For this reason, the process for the purification of the adipic acid has to be adapted in order to remove these impurities.

Besides, after the step of oxidation of the compounds cyclohexanone and cyclohexanol by nitric acid, the adipic acid is purified, notably during a step of crystallization so as to obtain adipic acid crystals. The crystallization of adipic acid is carried out starting from the solutions obtained during the oxidation of cyclohexanone/cyclohexanol mixtures by nitric acid. These solutions are acidic and comprise a large amount of nitric acid and/or nitrate ions.

With stainless steel materials, phenomena of fouling of crystallization devices by deposition of crystalline adipic acid on the walls of the equipment have been remarked. This phenomenon, known as “fouling”, depends on the nature of the material and on the surface condition of the walls.

Because of this fouling it is necessary to periodically shut down the process in order to remove the deposits of adipic acid on the walls of the heat exchanger or the walls of the crystallizer.

The surface condition is measured notably by the surface roughness of the material for example according to the method defined by Standards NF EN ISO 3274 and NF EN ISO 4288. The more the roughness value is important, the more the fouling phenomenon is increased.

One of the aims of the present invention is to overcome these disadvantages by proposing to carry out the stage of manufacture of the adipic acid, namely the oxidation of the compounds cyclohexanone and cyclohexanol by nitric acid, in devices and reactors constructed of a material which limits the corrosion phenomenon and which thus reduces the presence of impurities in the adipic acid produced. Another aim is to limit the fouling phenomenon of the crystallization devices walls by adipic acid.

To this end, the invention provides a process for the manufacture of adipic acid comprising a stage of oxidation of cyclohexanol/cyclohexanone compounds by nitric acid carried out in a plant comprising at least one reactor and devices or apparatuses, characterized in that at least the parts of the reactor in contact with the reaction medium for nitric acid oxidation and optionally parts of the devices and/or apparatuses are made with a “duplex” austenoferritic stainless steel material of X2 CrNiN 23-4 (1.4362) type according to the European nomenclature. These steels can be marketed under the trade names Uranus 35N or SAF 2304.

The expression “at least the walls in contact with the reaction media” should be understood as meaning that the devices or reactors can be made entirely with “duplex” austenoferritic stainless steel of X2 CrNiN 23-4 type or only partially with this material. In the latter case, the parts made of “duplex” austenoferritic stainless steel of X2 CrNiN 23-4 type are those coming into contact with the reaction medium or the vapours and gases originating from this reaction medium.

Thus, it has been found that the phenomenon of corrosion of the walls or parts made of “duplex” austenoferritic stainless steel of X2 CrNiN 23-4 type is absent or very limited in comparison with the phenomenon observed with other stainless steels marketed as resistant to corrosion by nitric acid, such as, for example, austenitic stainless steel of AISI 304L type.

The “duplex” austenoferritic stainless steel of X2 CrNiN 23-4 type is a material having a composition defined in Standards NF EN 10088-1 (former NFA35-574) and NF EN 10028-7, which are applicable in their latest revision.

The medium of oxidation of cyclohexanol and/or cyclohexanone by nitric acid comprises metal compounds.

The medium for the oxidation of cyclohexanol and/or cyclohexanone by nitric acid comprises nitric acid at a concentration advantageously of between 20 and 35% by weight, metal compounds forming the nitric acid oxidation catalyst and metal compounds present in the cyclohexanol and/or cyclohexanone and corresponding to the catalysts used in the oxidation of cyclohexane by oxygen or the decomposition of hydroxycyclohexyl peroxide.

The nitric acid oxidation reaction is carried out at a temperature of between 70 and 120° C. and advantageously between 70 and 100° C., generally under an absolute pressure of between 1 bar and 5 bars.

According to the invention, the reactors used for carrying out the nitric acid oxidation are constructed of “duplex” austenoferritic stainless steel of X2 CrNiN 23-4 type. It is also advantageous to produce the other apparatuses and devices of the plant with this “duplex” austenoferritic stainless steel material of X2 CrNiN 23-4 type.

Advantageously, the process according to the invention comprises a step of crystallization of the adipic acid, the devices or apparatuses comprise a crystallization vessel equipped with means for stirring and means for cooling and/or concentrating the adipic acid solution and at least a part of the walls of the crystallization vessel and/or of the means for cooling and/or concentrating in contact with the adipic acid solution is made of “duplex” austenoferritic stainless steel material of X2 CrNiN 23-4 type (1.4362) according to the European nomenclature.

Mention may also be made, as examples, as other devices and apparatuses advantageously made of “duplex” austenoferritic stainless steel of X2 CrNiN 23-4 type, of all or parts of the reactors, finishers, nitric acid concentrating columns, or the like. This list does not have a limiting nature and is given solely by way of indication. This is because all the devices of the plant for the manufacture of adipic acid in contact with a medium comprising nitric acid can be made of “duplex” austenoferritic stainless steel of X2 CrNiN 23-4 type.

The invention will be better illustrated in the light of the examples given below solely by way of indication.

The corrosion tests given below solely by way of illustration clearly show the advantages introduced by the use of “duplex” austenoferritic stainless steel of X2 CrNiN 23-4 type, in the process for the manufacture of adipic acid by oxidation of cyclohexanol and/or cyclohexanone by nitric acid.

Tests for determining the resistance to corrosion and the change in the surface condition of items made of different grades of stainless steel were carried out according to the procedure below:

Test specimens of parallelepipedal shape with dimensions of 50×30 mm, the surface of which was polished in order to have an initial roughness Ra of less than 0.1 μm, were immersed in a medium resulting from the oxidation of a cyclohexanone/cyclohexanol mixture by nitric acid comprising a concentration by weight of adipic acid of 24% and a content of nitric acid of the order of 28%.

The solution is maintained at a temperature of 90° C. under atmospheric pressure and is stirred throughout the duration of immersion. After immersing for 400 hours, the surface condition of the test specimens and the loss in thickness are determined. These test specimens are again immersed in the same medium for a further duration of 400 hours. However, the solution is replaced before each further immersion.

The test specimens tested were made of two grades of stainless steel:

Test specimen 1: steel of AISI 304L type

Test specimen 2: steel of X2 CrNiN 23-4 type (Uranus 35N or SAF2304 equivalent).

The compositions of these grades of steel are given in Table I below:

| | | |
|---|---|---|
| Metal | 304L | X2 CrNiN 23-4 |

| | |
|---|---|
| composition | % by weight |

| | | |
|---|---|---|
| C | 0.015 | 0.033 |
| S | 0.002 | 0.001 |
| P | 0.025 | 0.023 |
| Si | 0.248 | 0.505 |
| Mn | 1.69 | 1.37 |
| Cr | 18.41 | 23.68 |
| Ni | 10.48 | 4.16 |
| Mo | 0.125 | 0.264 |
| Cu | 0.069 | 0.175 |
| N | — | 0.12 |
| Fe | bal. | bal. |

(bal. means remainder to 100%)

The results observed are collated in Table II below:

| Test specimen | | 1 | 2 |
|---|---|---|---|
| Initial | Roughness Ra (μm) | <0.1 | <0.1 |
| | Loss in thickness | 0 | 0 |
| | (μm/year) | | |
| 400 hours | Roughness Ra (μm) | 0.5 | 1.3 |
| | Loss in thickness | 90 | 160 |
| | (μm/year) | | |
| 800 hours | Roughness Ra (μm) | 1.9 | 1.7 |
| | Loss in thickness | 130 | 90 |
| | (μm/year) | | |
| 1200 hours | Roughness Ra (μm) | 2.8 | 1.7 |
| | Loss in thickness | 130 | 60 |
| | (μm/year) | | |
| 1600 hours | Roughness Ra (μm) | 4.1 | 1.8 |
| | Loss in thickness | 230 | 70 |
| | (μm/year) | | |
| 2000 hours | Roughness Ra (μm) | 4.7 | 2.1 |
| | Loss in thickness | 90 | 60 |
| | (μm/year) | | |
| 2400 hours | Roughness Ra (μm) | 4.7 | 2.1 |
| | Loss in thickness | 140 | 80 |
| | (μm/year) | | |

It emerges from the above results that, for the test specimen 2, which corresponds to the present invention, there is a slight variation of the roughness upon time compared to the test specimen measured for a AISI 304L type (test specimen 1). This feature shows the fact that the use of a steel of X2CrNiN23-4 type according to the invention allows to maintain a good surface condition upon time for the concerned reaction and thus to limit the phenomenon of fouling as much as possible.

Moreover, the above results show that the resistance to corrosion (loss in thickness) of a steel of X2CrNiN23-4 type in a medium resulting from the oxidation of a cyclohexanone/cyclohexanol mixture by nitric acid (according to the invention) is increased.

1. A process for the manufacture of adipic acid, the process comprising oxidizing cyclohexanol and/or cyclohexanone by nitric acid wherein the process is carried out in a plant comprising at least one reactor and a device or apparatus, wherein at least parts of the reactor that come into contact with a reaction medium for nitric acid oxidation and optionally parts of the device and/or apparatus are made with a “duplex” austenoferritic stainless steel X2 CrNiN 23-4 (1.4362) material, according to the European nomenclature.

2. The process according to claim 1, wherein the reactor or reactors are made of “duplex” austenoferritic stainless steel X2 CrNiN 23-4 (1.4362) material.

3. The process according to claim 1, wherein the reaction medium for nitric acid oxidation comprises a metal compound.

4. The process according to claim 1, wherein the nitric acid oxidation reaction is carried out at a temperature between 70° C. and 120° C.

5. The process according to claim 1, wherein the device of the plant in contact with the reaction medium for nitric acid oxidation is made of “duplex” austenoferritic stainless steel X2 CrNiN 23-4 (1.4362) material.

6. The process according to claim 1, wherein the process further comprises a step of crystallizing the adipic acid, in that the device or apparatus comprise a crystallization vessel equipped with a means for stirring and a means for cooling and/or concentrating the adipic acid solution and in that at least a part of the walls of the crystallization vessel and/or of the means for cooling and/or concentrating that comes into contact with the adipic acid solution is made of “duplex” austenoferritic stainless steel X2 CrNiN 23-4 (1.4362) material, according to the European nomenclature.
